(19) United States
(12) Patent Application Publication
(10) Pub. No.: US 2009/0233898 A1
(43) Pub. Date: Sep. 17, 2009

(54) Pharmaceutical Compositions Comprising Simvastatin and Ezetimibe

(75) Inventors: Kamal Mehta, Rajasthan (IN); Abhay Mahajan, Maharashtra (IN); Pankaj Sinde, Maharashtra (IN)

Correspondence Address: GLENMARK PHARMACEUTICALS INC USA, 750 CORPORATE DRIVE, MAHWAH, NJ, 07430, US

(73) Assignee: GLENMARK GENERICS LTD, Mumbai (IN)

(21) Appl. No.: 12/400,954

(22) Filed: Mar. 10, 2009

Related U.S. Application Data
(60) Provisional application No. 61/132,912, filed on Jun. 24, 2008.

(30) Foreign Application Priority Data
Mar. 11, 2008 (IN) 493/MUM/2008

(51) Int. Cl. A61K 31/397; A61P 9/10

(52) U.S. Cl. 514/210.02

(57) ABSTRACT

The present invention generally relates to a stable pharmaceutical composition that does not have a film coating which contains simvastatin, ezetimibe or pharmaceutically-acceptable salts thereof, and ascorbic acid.

### PRIORITY

This application claims priority to Indian Provisional Application No. 493/MUM/2008, filed on Mar. 11, 2008, and under U.S.C. §119 (c) to U.S. Provisional Application No. 61/132,912, filed on Jun. 24, 2008; the contents of which are hereby incorporated by reference.

### BACKGROUND OF THE INVENTION

1. Technical Field

The present invention generally relates to a pharmaceutical composition comprising an HMG-CoA reductase inhibitor and a cholesterol absorption inhibitor or a pharmaceutically-acceptable salt thereof and ascorbic acid

2. Description of the Related Art

In general, simvastatin is a hypolipidemic drug belonging to the class of pharmaceuticals called HMG-CoA reductase inhibitors or “statins”. Simvastatin has the structure, as shown below:

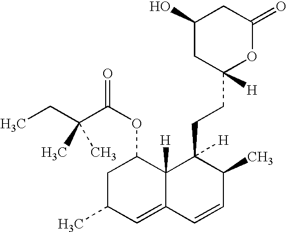

Generally, simvastatin is a synthetic lipid-lowering agent that acts as an inhibitor of 3-hydroxy-3-methylglutaryl-coenzyme A (HMG-CoA) reductase (HMG-CoA Reductase inhibitor). This enzyme catalyzes the conversion of HMG-CoA to mevalonate, an early and rate-limiting step in cholesterol biosynthesis.

HMG-CoA reductase inhibitors are commonly referred to as “statins.” Statins are therapeutically effective drugs used for reducing low density lipoprotein (LDL) particle concentration in the blood stream of patients at risk for cardiovascular disease. Simvastatin is indicated for use for reducing elevated total cholesterol (total-C), low-density lipoprotein cholesterol (LDL-C), apolipoprotein B (Apo B), and high plasma triglycerides (TG), and to increase high-density lipoprotein cholesterol (HDL-C) in patients with primary hypercholesterolemia (heterozygous familial and nonfamilial) and mixed dyslipidemia (Fredrickson types IIa and IIb4); treating patients with hypertriglyceridemia (Fredrickson type IV hyperlipidemia); treating patients with primary dysbetalipoproteinemia (Fredrickson type III hyperlipidemia), and reducing total-C and LDL-C in patients with homozygous familial hypercholesterolemia as an adjunct to other lipid-lowering treatments (e.g., LDL apheresis).

Ezetimibe is a selective cholesterol absorption inhibitor that effectively blocks intestinal absorption of dietary and biliary cholesterol and is represented by the structure as shown below:

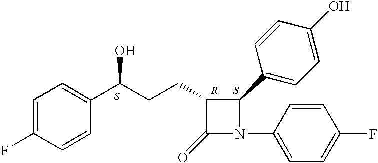

Ezetimibe undergoes glucuronidation to a single metabolite and is localized in the intestinal wall, where it prevents cholesterol absorption. Enterohepatic recirculation of ezetimibe and the glucoronide ensures repeated delivery to the site of action and limits peripheral exposure. Ezetimibe does not affect the absorption of fat-soluble vitamins or triglycerides.

The combination of simvastatin and ezetimibe is also available in the United States under the trade name VYTORIN® marketed by Merck.

U.S. Pat. No. 7,229,982 (“the '982 patent”) and U.S. Patent Application Publication No. 20070160666 (“the '666 application”) discloses that citric acid alone can replace the combination of ascorbic acid and citric acid (2:1) to form a stable composition and the removal of ascorbic acid, eliminates the problem of discoloration and also eliminates the need of film coat over the tablets.

WO2007003365 (“the '365 PCT application”) discloses a pharmaceutical composition of simvastatin and ezetimibe that do not contain any stabilizing agents particularly antioxidants. The '365 PCT application also discloses that the contact between the pharmaceutical composition and oxygen containing environment is reduced by use of coatings or packing under reduced pressure or inert gas atmosphere.

### SUMMARY OF THE INVENTION

The present invention provides a pharmaceutical composition comprising simvastatin, or a pharmaceutically acceptable salt thereof together, ezetimibe, or a pharmaceutically acceptable salt thereof.

The present invention provides a pharmaceutical composition comprising simvastatin, or a pharmaceutically acceptable salt thereof, ezetimibe, or a pharmaceutically acceptable salt thereof, and at least one stabilizer.

The present invention provides a pharmaceutical composition comprising simvastatin, or a pharmaceutically acceptable salt thereof, and ezetimibe, or a pharmaceutically acceptable salt thereof, wherein ascorbic acid is one of the stabilizers.

The present invention provides a pharmaceutical composition comprising simvastatin, or a pharmaceutically acceptable salt thereof, and ezetimibe, or a pharmaceutically acceptable salt thereof, wherein ascorbic acid and BHA are present as stabilizers.

The present invention provides a pharmaceutical composition comprising simvastatin, or a pharmaceutically acceptable salt thereof, and ezetimibe, or a pharmaceutically acceptable salt thereof, wherein ascorbic acid, BHA and citric acid are present as stabilizers.

The present invention provides a pharmaceutical composition in tablet form comprising simvastatin, or a pharmaceutically acceptable salt thereof and ezetimibe or a pharmaceutically acceptable salt thereof, which contains no more than 5% of ascorbic acid as a stabilizer.

The present invention provides a pharmaceutical composition in tablet form comprising simvastatin, or a pharmaceutically acceptable salt thereof, and ezetimibe or a pharmaceutically acceptable salt thereof, which contains no more than 5% of ascorbic acid as a stabilizer, wherein the tablet does not have a film coating.

### DETAILED DESCRIPTION OF THE INVENTION

It has previously been found that ascorbic acid used in a composition containing simvastatin and ezetimibe causes a discoloration of the final the composition. To overcome this problem, a film coating has been deemed to be essential. However, such an option is expensive, and accordingly, the prior formulations avoided the use of ascorbic acid.

The inventors of the present invention have surprisingly found that, contrary to prior findings, by carefully controlling the amount of ascorbic acid, which can be used alone or in combination with butylated hydroxyanisole (“BHA”) and/or citric acid, eliminates the problem of discoloration and eliminates the need for coating such compositions.

The present invention is directed to a pharmaceutical composition containing simvastatin, ezetimibe and ascorbic acid as a stabilizer. The pharmaceutical composition of the present invention is stable without requiring a film coating.

The present invention is also directed to process of making pharmaceutical composition by direct compression and/or by wet granulation and/or by dry granulation process.

The pharmaceutical compositions of the present invention may contain one or more pharmaceutically acceptable excipients. Suitable pharmaceutically acceptable excipients include, but are not limited to, diluents, disintegrants, binders, lubricants and the like and mixtures thereof.

Suitable diluents include, but are not limited to, lactose, dicalcium phosphate, calcium sulfate, kaolin, mannitol, sorbitol, inositol, sodium chloride, dry starch, powdered sugar and the like and mixtures thereof.

Suitable disintegrants include, but are not limited to, carboxymethylcellulose calcium, carboxymethylcellulose sodium (e.g., Ac-Di-Sol®, Primellose croscarmellose sodium), sodium starch glycolate (e.g. Explotab®), crospovidone, guar gum, magnesium aluminum silicate, methyl cellulose, polacrilin potassium, powdered cellulose, pregelatinized starch, sodium alginate and starch, and the like and mixtures thereof wherein sodium starch glycolate is most preferred.

Suitable binders include, but are not limited to, polyvinylpyrrolidone, starch mucilage, pregelatinized starch, sodium alginate, alginic acid, acacia mucilage, tragacanth, hydroxypropyl methyl cellulose, carboxymethylcellulose sodium, carboxymethylcellulose calcium, ethyl cellulose, polyethylene glycol, hydroxyethyl cellulose, hydroxypropyl cellulose, methyl cellulose, polymethacrylate, carboxyvinyl polymers such as carbopols and the like and mixtures thereof.

Suitable lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, zinc stearate and the like and mixtures thereof.

Suitable glidants include, but are not limited to, talc, colloidal silicon di oxide and the like and mixtures thereof.

Suitable “antioxidants” as stabilizers include, but are not limited to, BHA, ascorbic acid, citric acid and mixtures thereof.

The pharmaceutical compositions of present invention can be prepared with techniques well known in the art, preferably, direct compression, dry granulation and wet granulation.

Actual dosage levels of the novel strontium salt of esomeprazole of the present invention may be varied to obtain an amount that is effective to obtain a desired therapeutic response for a particular composition and method of administration for treatment of a mammal. The selected dosage level therefore depends upon such factors as, for example, the desired therapeutic effect, the route of administration, the desired duration of treatment, and other factors. The total daily dose of the composition of the present invention can be administered to a host in single or divided dose and can vary widely depending upon a variety of factors including, for example, the body weight, general health, sex, diet, time and route of administration, rates of absorption and excretion, combination with other drugs, the severity of the particular condition being treated, etc.

The following examples are provided to enable one skilled in the art to practice the invention and are merely illustrative of the invention. The examples should not be read as limiting the scope of the invention as defined in the features and advantages.

### EXAMPLES

### Example 1

| Sr. | | Qty/Tab. |
|---|---|---|
| No. | Ingredient | (in mg) |
| Dry Mix | | |
| 1. | Ezetimibe | 10.00 |
| 2. | Simvastatin | 80.00 |
| 3. | Lactose Monohydrate | 596.34 |
| 4. | Microcrystalline Cellulose | 50.00 |
| | (MCC) | |
| 5. | Sodium Starch Glycolate | 5.00 |
| 6. | Hypromellose | 16.00 |
| 7. | BHA | 0.16 |
| 8. | Ascorbic acid | 20.00 |
| 9. | Purified water | qs |
| 10. | Isopropyl Alcohol (IPA) | qs |
| Lubrication | | |
| 11. | Sodium starch glycolate | 2.50 |
| 12. | Colloidal silicon dioxide | 4.00 |
| 13. | Talc | 4.00 |
| 14. | Magnesium Stearate | 12.00 |

| | |
|---|---|
| Total weight | 800.00 |

Procedure

1. Ezetimibe and a portion of lactose sifted through ASTM mesh #40.

2 Simvastatin, lactose, sodium starch glycolate, ascorbic acid, hypromellose and MCC sifted through ASTM mesh #40.

3. The above sifted blend is added to RMG and dry mixed for 10 minutes.

4, BHA is then dissolved in IPA under stirring; to it purified water was added to get the granulating solvent.

5. The dry mix of step No. 3. was granulated with the above prepared granulating solution and granules were dried at 50 to 60° C. for 60 minutes to achieve LOD NMT 2.0%.

6. The dried granules were then sifted through ASTM mesh No. #20.

7, Sodium starch glycolate, colloidal silicon dioxide and talc in extra were first blended with the sifted granules of step No. 7 for 5 minutes.

8. Magnesium stearate, passed through ASTM mesh #60, was then added to the granules of step No. 8 and mixed for 5 minutes.

This blend was then compressed into the tablets.

### Example 2

| | | |
|---|---|---|
| Sr. | | Qty/Tab. |
| No. | Ingredient | (in mg) |

Dry Mix

| | | |
|---|---|---|
| 1. | Ezetimibe | 10.00 |
| 2. | Simvastatin | 80.00 |
| 3. | Lactose Monohydrate | 596.34 |
| 4. | Microcrystalline | 50.00 |
| | Cellulose | |
| 5. | Sodium Starch Glycolate | 5.00 |
| 6. | Hypromellose | 16.00 |
| 7. | BHA | 0.16 |
| 8. | Purified water | qs |
| 9. | Isopropyl Alcohol | qs |

Lubrication

| | | |
|---|---|---|
| 10. | Sodium starch glycolate | 2.50 |
| 11. | Ascorbic acid | 20.00 |
| 12. | Colloidal silicon dioxide | 4.00 |
| 13. | Talc | 4.00 |
| 14. | Magnesium Stearate | 12.00 |

| | |
|---|---|
| Total Weight | 800.00 |

Procedure

1. Ezetimibe and a portion of lactose were sifted through ASTM mesh #40.

2. Simvastatin, lactose, sodium starch glycolate, hypromellose and microcrystalline cellulose sifted through ASTM mesh #40.

3. The above sifted blend was added to RMG and dry mixed for 10 minutes.

4. BHA was dissolved in IPA under stirring; to it purified water was added to get the granulating solvent.

5. The dry mix of step No, 3. was granulated with the above prepared granulating solution and granules were then dried at 50 to 60° C. for 60 minutes to achieve LOD NMT 2.0%.

6. The dry granules were sifted through ASTM mesh no. #20-#24.

7. Sodium starch glycolate, acorbic acid, colloidal silicon dioxide and talc in extra were first blended with the sifted granules of step No. 6 for 5 minutes.

8. Magnesium stearate, passed through ASTM mesh #60, was added to the granules of step No. 7 for 5 minutes.

9. This blend was then compressed into the tablets.

### Example 3

| Sr. | | Qty/Tab. |
|---|---|---|
| No. | Ingredient | (in mg) |
| Dry Mix | | |
| 15. | Ezetimibe | 10.00 |
| 16. | Simvastatin | 80.00 |
| 17. | Lactose Monohydrate | 612.84 |
| 18. | Microcrystalline Cellulose | 50.00 |
| 19. | Sodium Starch Glycolate | 5.00 |
| 20. | Hypromellose | 16.00 |
| 21. | BHA | 0.16 |
| 22. | Ascorbic acid | 3.50 |
| 23. | Purified water^{@} | qs |
| 24. | Isopropyl Alcohol^{@} | qs |
| Lubrication | | |
| 25. | Sodium starch glycolate | 2.50 |
| 26. | Colloidal silicon dioxide | 4.00 |
| 27. | Talc | 4.00 |
| 28. | Magnesium Stearate | 12.00 |

| | |
|---|---|
| Total weight | 800.00 |

Procedure

1. Ezetimibe and a portion of lactose were sifted through ASTM mesh #40.

2. Simvastatin, lactose, sodium starch glycolate, ascorbic acid, hypromellose and MCC Sifted through ASTM mesh #40.

3. The above sifted blend is added to RMG and dry mixed for 10 minutes.

4. BHA is then dissolved in IPA under stirring; to it purified water was added to get the granulating solvent.

5. The dry mix of step No. 3. was granulated with the above prepared granulating solution and granules were dried at 50 to 60° C. for 60 minutes to achieve LOD NMT 2.0%.

6. The dried granules were then sifted through ASTM mesh No. #20.

7. Sodium starch glycolate, colloidal silicon dioxide and talc in extra were first blended with the sifted granules of step No. 7 for 5 minutes.

8. Magnesium stearate, passed through ASTM mesh #60, was then added to granules of step No. 8 and mixed for 5 minutes.

This blend was then compressed into the tablets

### Example 4

| Sr. | | Qty/Tab. |
|---|---|---|
| No. | Ingredient | (in mg) |
| Dry Mix | | |
| 29. | Ezetimibe | 10.00 |
| 30. | Simvastatin | 80.00 |
| 31. | Lactose Monohydrate | 615.34 |
| 32. | Microcrystalline Cellulose | 50.00 |
| 33. | Sodium Starch Glycolate | 5.00 |
| 34. | Hypromellose | 16.00 |
| 35. | BHA | 0.16 |
| 36. | Ascorbic acid | 1.00 |
| 37. | Purified water^{@} | qs |
| 38. | Isopropyl Alcohol^{@} | qs |
| Lubrication | | |
| 39. | Sodium starch glycolate | 2.50 |
| 40. | Colloidal silicon dioxide | 4.00 |
| 41. | Talc | 4.00 |
| 42. | Magnesium Stearate | 12.00 |

| | |
|---|---|
| Total weight | 800.00 |

Procedure

1. Ezetimibe and a portion of lactose were sifted through an ASTM mesh #40.

2. Simvastatin, Lactose, sodium starch glycolate, ascorbic acid, hypromellose and MCC were sifted through an ASTM mesh #40.

3. The above sifted blend is added to RMG and dry mixed for 10 minutes.

4. BHA is then dissolved in IPA under stirring; to it purified water was added to get the granulating solvent.

The dry mix of step No. 3. was granulated with the above prepared granulating solution and granules were dried at 50 to 60° C. for 60 minutes to achieve LOD NMT 2.0%.

The dried granules were then sifted through an ASTM mesh No. #20.

Sodium starch glycolate, colloidal silicon dioxide and talc in extra were first blended with the sifted granules of step No 7 for 5 minutes.

Magnesium stearate, passed through an ASTM mesh #60, was then added to granules of step No. 8 and mixed for 5 minutes.

This blend was then compressed into the tablets.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

1. A stable pharmaceutical composition that does not have a film coating comprising simvastatin and ezetimibe, or pharmaceutically acceptable salts thereof, and ascorbic acid, wherein the amount of ascorbic acid is less than 5 wt % of the final formulation.

2. The composition of claim 1, further comprising one or more additional stabilizers selected from BHA, citric acid and combinations thereof.
